(19) United States
(12) Patent Application Publication
(10) Pub. No.: US 2023/0181244 A1
(43) Pub. Date: Jun. 15, 2023

(54) SUPPORT ASSEMBLY FOR FLEXIBLE MEDICAL ASSEMBLY

(71) Applicant: Boston Scientific Medical Device Limited, Ballybrit, Galway (IE)

(72) Inventors: Christian Balkovec, Burlington (CA); Gareth Davies, Toronto (CA); John Paul Urbanski, Toronto (CA)

(21) Appl. No.: 17/924,212

(22) PCT Filed: Jun. 1, 2021
(86) PCT No.: PCT/IB2021/054816

§ 371 Date: Nov. 9, 2022

Related U.S. Application Data
(60) Provisional application No. 63/040,632, filed on Jun. 18, 2020.

(51) Int. Cl. A61B 18/14; A61M 25/01

(52) U.S. Cl. CPC A61B 18/1492; A61M 25/0136; A61M 25/0113; A61B 2018/1425; A61M 29/02

(57) ABSTRACT

An elongated support assembly is positionable, at least in part, in sliding relationship with an elongated flexible medical assembly. The elongated support assembly is configured to support, at least in part, the elongated flexible medical assembly after the elongated support assembly is positioned, at least in part, in sliding relationship with the elongated flexible medical assembly. The elongated support assembly is, at least in part, selectively maneuverable via an elongated ancillary medical assembly toward a distal portion of the elongated ancillary medical assembly.

### TECHNICAL FIELD

This document relates to the technical fields of (and is not limited to) (A) an elongated support assembly for an elongated flexible medical assembly and an elongated ancillary medical assembly, and method therefor; and/or (B) an elongated ancillary medical assembly for an elongated flexible medical assembly, and method therefor; and/or (C) a synergistic combination of an elongated support assembly, an elongated flexible medical assembly and an elongated ancillary medical assembly, and method therefor.

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) flexible medical assemblies (also called the existing technology). After much study of, and experimentation with, the existing (known) flexible medical assemblies, an understanding (at least in part) of the problem and its solution have been identified (at least in part) and are articulated (at least in part) as follows:

Puncturing the interatrial septum (a biological feature of the patient) may be performed during a transseptal catheterization procedure where access to the left atrium (of the heart) is achieved from the right atrium. A known stiff needle assembly (such as, a mechanical needle) may be used to puncture through the desired portion of the heart tissue. A known needle assembly having a radiofrequency emitter may be used for the case where it might be advantageous to avoid application of a mechanical force for forming the puncture through tissue. Radiofrequency-enabled needles may provide a safer, more reliable alternative to mechanical needles as the lack of input force required poses lower risk to creating inadvertent damage to the tissue (such as the heart) due to greater procedural control offered to the user. Radiofrequency energy may be used to vaporize tissue from an active electrode positioned at the distal tip of the needle (once the electrode is positioned proximate to, or in contact with, the tissue). However, once transseptal puncture via a radiofrequency needle is achieved, the user may be unable to instantly secure access in the left atrium (of the heart). Securing access may involve embedding a guidewire through the transseptal puncture site deep into the left atrium and into a pulmonary vein.

A known mechanical transseptal puncture needle has a hollow lumen that a guidewire may be loaded into, and deployed from, shortly after crossing the interatrial septum (of the heart). However, a known radiofrequency needle does not have a hollow lumen since the radiofrequency needle with a hollow lumen might inadvertently function very much like a hole punch, where a closed perimeter of electrically active and conductive material might vaporize tissue circumferentially around the distal profile of a lumen, thereby releasing an unwanted, free-floating, core of tissue into the blood stream. This free-floating core of tissue may be highly undesirable if it is permitted to free float in the bloodstream of the patient given that the core may present significant risk for stroke or pulmonary embolism.

It may be desirable to provide a device or a system that combines the reliability and safety of the known radiofrequency puncture with the ability to secure access in the left atrium of the patient once transseptal puncture is achieved.

To mitigate, at least in part, at least one problem associated with the existing technology, there is provided (in accordance with a major aspect) an apparatus. The apparatus is for use with an elongated flexible medical assembly and an elongated ancillary medical assembly. The apparatus includes and is not limited to (comprises) an elongated support assembly that is positionable, at least in part, in a sliding relationship with the elongated flexible medical assembly. The elongated support assembly is configured to support, at least in part, the elongated flexible medical assembly; this is done, preferably, after the elongated support assembly is positioned, at least in part, in a sliding relationship with the elongated flexible medical assembly. The elongated support assembly is, at least in part, selectively maneuverable via the elongated ancillary medical assembly toward a distal portion of the elongated ancillary medical assembly.

To mitigate, at least in part, at least one problem associated with the existing technology, there is provided (in accordance with a major aspect) an apparatus. The apparatus is for use with an elongated flexible medical assembly and an elongated ancillary medical assembly. The apparatus includes and is not limited to (comprises) an elongated support assembly that is positionable, at least in part, proximate to the elongated flexible medical assembly. The elongated support assembly is maneuverable, with the elongated flexible medical assembly positioned proximate to the elongated support assembly, along, at least in part, the elongated ancillary medical assembly toward a distal portion of the elongated ancillary medical assembly. The elongated support assembly is, at least in part, selectively extendable, with the elongated flexible medical assembly positioned proximate to the elongated support assembly, away from the distal portion of the elongated ancillary medical assembly. The elongated support assembly is configured to support, at least in part, the elongated flexible medical assembly while the elongated flexible medical assembly and the elongated support assembly are extended (in unison) away from, at least in part, the distal portion of the elongated ancillary medical assembly.

To mitigate, at least in part, at least one problem associated with the existing technology, there is provided (in accordance with a major aspect) a method. The method is for using an elongated flexible medical assembly, an elongated ancillary medical assembly and an elongated support assembly. The method includes and is not limited to (comprises) positioning, at least in part, the elongated support assembly in a sliding relationship with the elongated flexible medical assembly. The method also includes supporting, at least in part, the elongated flexible medical assembly via the elongated support assembly positioned, at least in part, in a sliding relationship with the elongated flexible medical assembly. The method also includes selectively maneuvering, at least in part, the elongated support assembly via the elongated ancillary medical assembly toward a distal portion of the elongated ancillary medical assembly.

To mitigate, at least in part, at least one problem associated with the existing technology, there is provided (in accordance with a major aspect) a method. The method is for using an elongated flexible medical assembly, an elongated ancillary medical assembly and an elongated support assembly. The method includes and is not limited to (comprises) positioning, at least in part, an elongated support assembly proximate to the elongated flexible medical assembly. The method also includes maneuvering the elongated support assembly, with the elongated flexible medical assembly positioned proximate to the elongated support assembly, along, at least in part, the elongated ancillary medical assembly toward a distal portion of the elongated ancillary medical assembly. The method also includes selectively extending, at least in part, the elongated support assembly, with the elongated flexible medical assembly positioned proximate to the elongated support assembly, away from the distal portion of the elongated ancillary medical assembly. The method also includes supporting, at least in part, the elongated flexible medical assembly with the elongated support assembly while the elongated flexible medical assembly and the elongated support assembly are extended (in unison) away from, at least in part, the distal portion of the elongated ancillary medical assembly.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 and FIG. 2 depict side views of embodiments of an elongated support assembly (for use with an elongated flexible medical assembly and an elongated ancillary medical assembly); and

FIG. 3 to FIG. 9 depict schematic cross-sectional side views of embodiments of the elongated support assembly of FIG. 1; and

FIG. 10 to FIG. 19B depict axial cross-sectional side views (FIG. 10 to FIG. 13A,

FIG. 14A, FIG. 14B, and FIG. 15A to FIG. 19B), radial cross-sectional side views (FIG. 13B and FIG. 13C), and a top view (FIG. 14C) of the embodiments of the elongated support assembly 102 of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

elongated support assembly 102
support lumen 104
biological feature 700
the pulmonary veins 702
ancillary medical assembly 800
distal portion 802
ancillary lumen 804
flexible medical assembly 900
distal puncture device 902
rotatable device 1000
arrow 1002
threads 1004
arrow 1006
handle 1100
portal 1102
arrow 1104
proximal hub 1200
arrow 1202
rotatable element 1300
arrow 1301
flexible element 1302
slidable element 1400
arrow 1402
proximal tapered section 1500
arrow 1502
flexible region 1600
arrow 1602
arrow 1604
block device 1700
arrow 1702
biasing device 1800
stopper 1802
depression device 1804
arrow 1806
arrow 1808
actuatable plunger 1900
arrow 1902

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

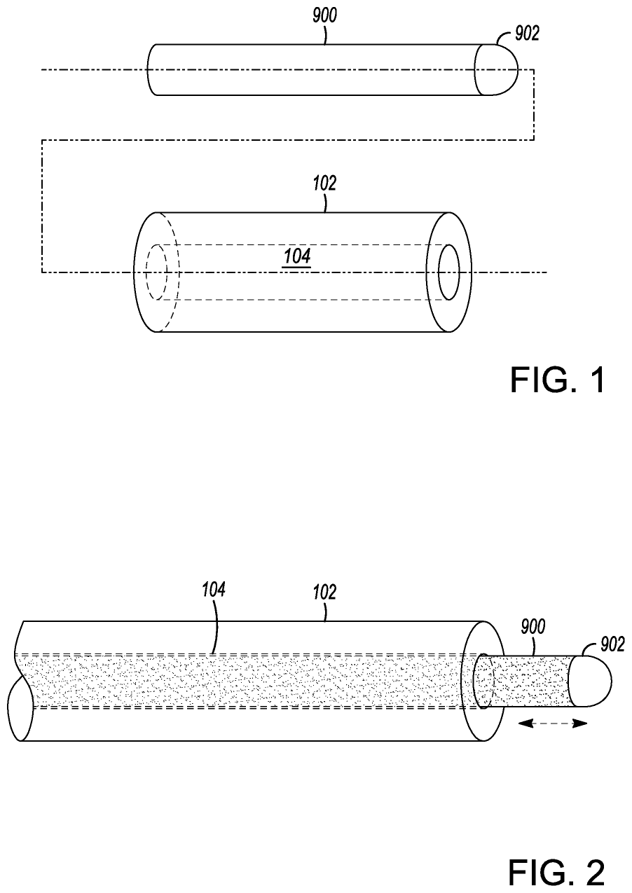

FIG. 1 and FIG. 2 depict side views of embodiments of an elongated support assembly 102 (for use with an elongated flexible medical assembly 900 and an elongated ancillary medical assembly 800).

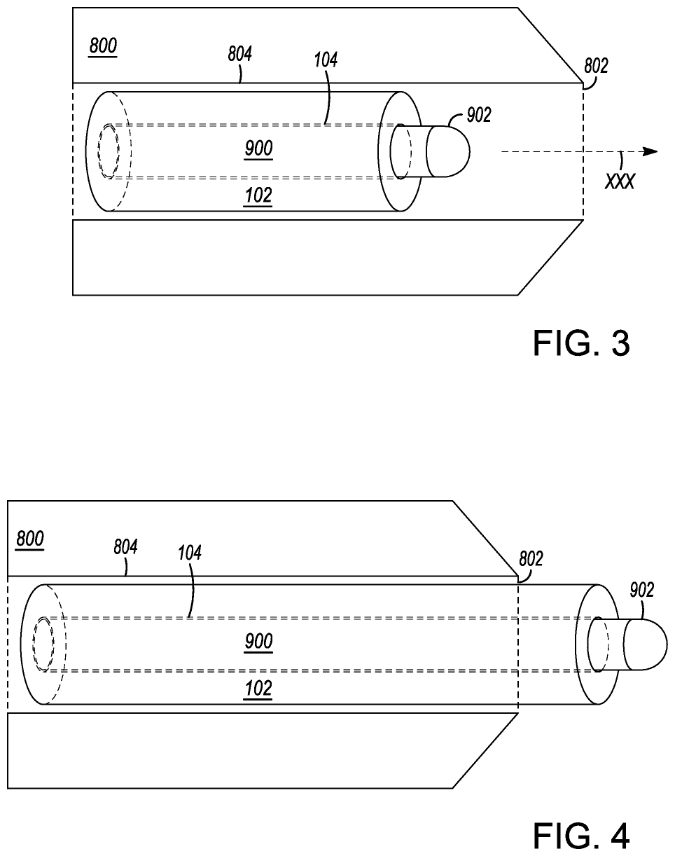

FIG. 3 to FIG. 9 depict schematic cross-sectional side views of embodiments of the elongated support assembly 102 of FIG. 1.

Referring to the embodiments as depicted in FIG. 1 and FIG. 2, the elongated support assembly 102 includes, preferably, a hollow tube, a hypotube, a guidewire defining a lumen, a hollow member, etc., and any equivalent thereof. The elongated support assembly 102 includes an elongated hollow tube with a curved outer surface. The elongated support assembly 102 defines, preferably, a support lumen 104 (also called an elongated support lumen) extending, at least in part, along a longitudinal length of the elongated support assembly 102. The support lumen 104 is configured to receive (slidably receive), at least in part, the elongated flexible medical assembly 900. The elongated support assembly 102 includes, preferably, a material comprising a stainless steel alloy and/or a nitinol alloy.

The elongated support assembly 102 may have two regions of differing stiffness. The region of the elongated support assembly 102 that is received within the ancillary lumen 804 proximate to the curved section at the distal end of the ancillary medical assembly 800 may be stiffer than the region of the elongated support assembly 102 at the curved region of the ancillary medical assembly 800 and beyond. This is to prevent distortion or disruption of the curvature present at the distal end of the ancillary medical assembly 800. The elongated support assembly 102 includes a first region having a first stiffness. The first region of the elongated support assembly 102 is configured to be positioned within the ancillary lumen 804 located proximate to a curved section at a distal end of the ancillary medical assembly 800. The elongated support assembly 102 includes a second region having a second stiffness. The second region of the elongated support assembly 102 is configured to be positioned at a curved region of the ancillary medical assembly 800 and beyond. The first region of the elongated support assembly 102 is relatively stiffer than the second region of the elongated support assembly 102 in such a way that distortion of any curvature at the distal end of the ancillary medical assembly 800 is prevented AFTER (A) the first region of the elongated support assembly 102 is positioned within the ancillary lumen 804 located proximate to a curved section at a distal end of the ancillary medical assembly 800, and (B) the second region of the elongated support assembly 102 is positioned at a curved region of the ancillary medical assembly 800 and beyond.

Referring to the embodiments as depicted in FIG. 1 and FIG. 2, the elongated support assembly 102 may include laser-cut features extending along a longitudinal length of the elongated body of the elongated support assembly 102. The laser-cut features may be formed by removal of material from a side wall of a hypotube resulting in a lower degree of stiffness or a higher degree of flexibility of the elongated support assembly 102.

The laser-cut features may aid in increasing or decreasing the degree of flexibility of the elongated support assembly 102.

Referring to the embodiments as depicted in FIG. 1 and FIG. 2, the elongated support assembly 102 may include helically-wound metal strands surrounding the support lumen 104. The helically-wound metal strands may provide greater flexibility in comparison to a single continuous piece of material.

Referring to the embodiments as depicted in FIG. 1 and FIG. 2, the elongated support assembly 102 may have a maximum outer diameter of about 0.032 inches to about 0.035 inches. The elongated support assembly 102 may have a minimum outer diameter of about 0.014 inches to about 0.024 inches. The elongated support assembly 102 is configured to be (preferably) received (slide received) within the ancillary medical assembly 800. The elongated support assembly 102 may include any suitable material that might conform to (the shape of the interior of) the ancillary medical assembly 800 without excessive geometric deformation of the elongated support assembly 102. The ancillary medical assembly 800 is configured to be inserted into a confined space defined by the patient.

Referring to the embodiments as depicted in FIG. 1 and FIG. 2, the elongated support assembly 102 includes, preferably, biocompatible material properties suitable for sufficient performance (such as dielectric strength, thermal performance, electrical insulation, corrosion, water resistance, heat resistance, etc.) for compliance with industrial and regulatory safety standards (or compatible for medical usage),etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiments as depicted in FIG. 1 and FIG. 2, the elongated support assembly 102 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus applied to the shape-memory material. This is known as the shape memory effect (SME). Superelasticity (in alloys) may be observed once the shape-memory material is deformed under the presence (an application) of a stimulus force.

Referring to the embodiments as depicted in FIG. 1 and FIG. 2, the elongated support assembly 102 is configured to be utilized in cooperation with the elongated ancillary medical assembly 800. The elongated support assembly 102 may include a metallic alloy configured to impart a degree of overall stiffness to the ancillary medical assembly 800 that may enhance aspects of the workflow for a given procedure. The elongated support assembly 102 may be, preferably, compatible with the minimum characteristics of the ancillary medical assembly 800.

Referring to the embodiments as depicted in FIG. 1 and FIG. 2, the elongated ancillary medical assembly 800 may include a transseptal accessory device, a sheath assembly, a dilator assembly, etc., and any equivalent thereof. The elongated ancillary medical assembly 800 defines, preferably, an ancillary lumen 804 extending, at least in part, along a longitudinal length of the elongated ancillary medical assembly 800.

Referring to the embodiments as depicted in FIG. 1 and FIG. 2, the elongated flexible medical assembly 900 may include a distal puncture device 902 configured to puncture a biological feature 700 (such as the interatrial septum of the heart of the patient). The elongated flexible medical assembly 900 may include an elongated needle assembly, etc., and any equivalent thereof. The distal puncture device 902 may include a radiofrequency puncture device. After performing a medical function (such as formation of a puncture through a biological feature or wall), the elongated flexible medical assembly 900 may be advanced through the elongated support assembly 102 (for various purposes, such as securing access to the left atrium of the heart of the patient). Therefore, in accordance with an aspect, the elongated support assembly 102 may be utilized for performing a medical function, such as puncturing the interatrial septum (of the heart of the patient) during a transseptal catheterization procedure. It will be appreciated that any configuration and/or construct of the elongated support assembly 102 may be utilized to facilitate embedding within the left atrium immediately following puncture to secure access.

Referring to the embodiments as depicted in FIG. 1 and FIG. 2, the elongated flexible medical assembly 900 may include (and is not limited to) a radiofrequency puncture device, such as the BAYLIS (TRADEMARK) POWERWIRE (REGISTERED TRADEMARK) radiofrequency guidewire manufactured by BAYLIS MEDICAL COMPANY (headquartered in Canada). In accordance with another embodiment, the flexible medical assembly 900 includes (and is not limited to) an elongated guidewire having a distal tip section presenting a mechanical cutting portion, etc.

Referring to the embodiments as depicted in FIG. 1 and FIG. 2, the elongated support assembly 102 has an outer diameter that is compatible with the inner diameter of the ancillary medical assembly 800. The outer diameter of the flexible medical assembly 900 (and/or the distal puncture device 902) has a maximum outer diameter that does not exceed the inner diameter of the elongated support assembly 102.

Referring to the embodiments as depicted in FIG. 1 and FIG. 2, the distal puncture device 902 has, preferably, a stainless steel core and/or a nitinol core, with a polytetrafluoroethylene (PTFE) heat shrink insulation jacket. The distal puncture device 902 includes a distal electrode that has a dome shape. The distal puncture device 902 has, preferably, a maximum outer diameter of about 0.014 inches to about 0.24 inches. The distal puncture device 902 may include any suitable conductive material as part of the core of a radiofrequency puncture device, etc. The distal puncture device 902 may include any suitable electrically insulative material to insulate a conductive core of a radiofrequency puncture device. The distal puncture device 902 has, preferably, a maximum outer diameter that is compatible with the minimum inner diameter of the elongated support assembly 102.

Referring to the embodiments as depicted in FIG. 1 and FIG. 2, the distal puncture device 902 may be usable with articulating distal elements (known and not depicted). The flexible medical assembly 900 may be used to puncture the interatrial septum, and then may be advanced into the left atrium (of the heart) further following puncturing of tissue, while using the articulating elements to change the distal conformation of the flexible medical assembly 900 from a straight, continuous geometry to a geometry that is not straight and continuous as the flexible medical assembly 900 may be bent at various articulation sites (if desired).

Referring to the embodiments as depicted in FIG. 1 and FIG. 2, the flexible medical assembly 900 may include an expandable-and-contractible structure (such as, a cage, a balloon, etc., and any equivalent thereof) positioned at (mounted to) a distal section of the flexible medical assembly 900. The expandable-and-contractible structure is configured to contact (at least in part) a biological feature (such as the interatrial septum). The expandable-and-contractible structure is configured to tightly compress against the body of the flexible medical assembly 900. Following crossing into the left atrium, the expandable-and-contractible structure is configured to expand and prevent (at least in part) access from being lost.

Referring to the embodiments as depicted in FIG. 1 and FIG. 2, the elongated support assembly 102 receives the flexible medical assembly 900. The inner diameter of the elongated support assembly 102 is compatible with the outer diameter of the flexible medical assembly 900.

Referring to the embodiments as depicted in FIG. 3 to FIG. 6, the elongated support assembly 102 and the flexible medical assembly 900 are positioned inside the ancillary medical assembly 800. The outer diameter of the elongated support assembly 102 is compatible with the inner diameter of the ancillary medical assembly 800.

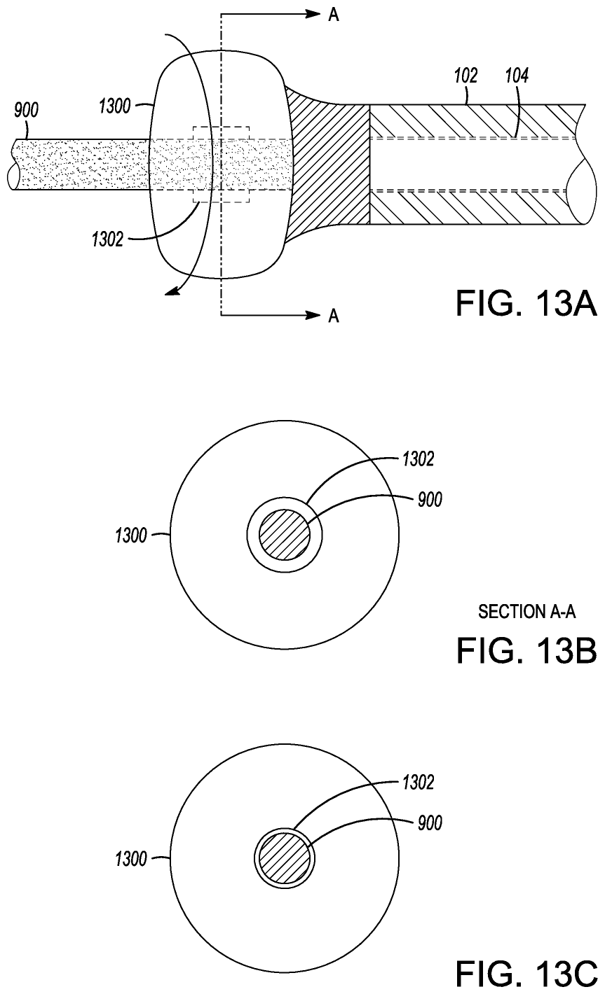

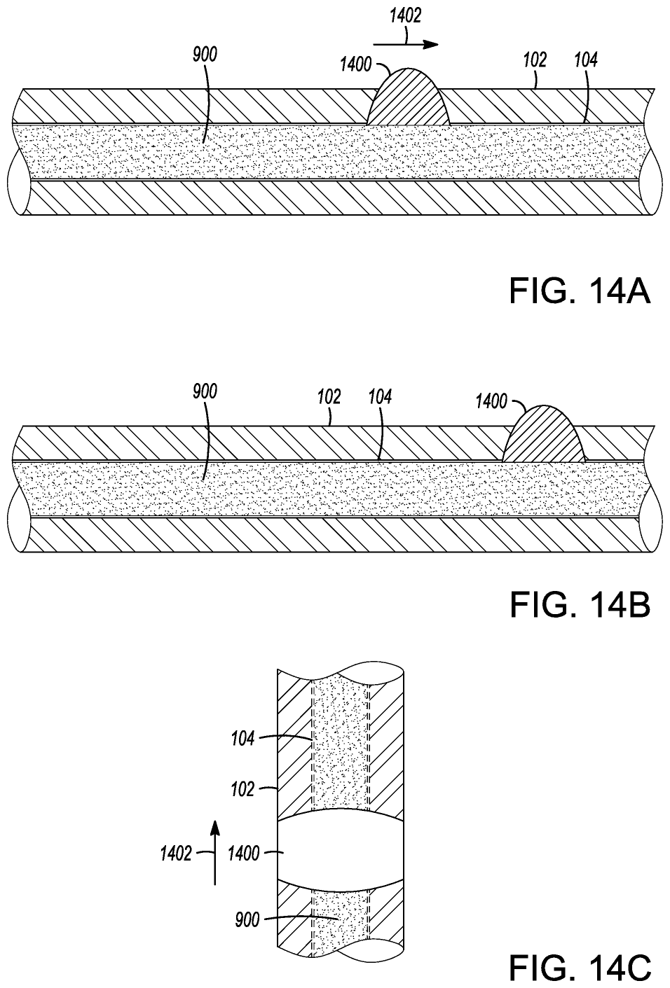

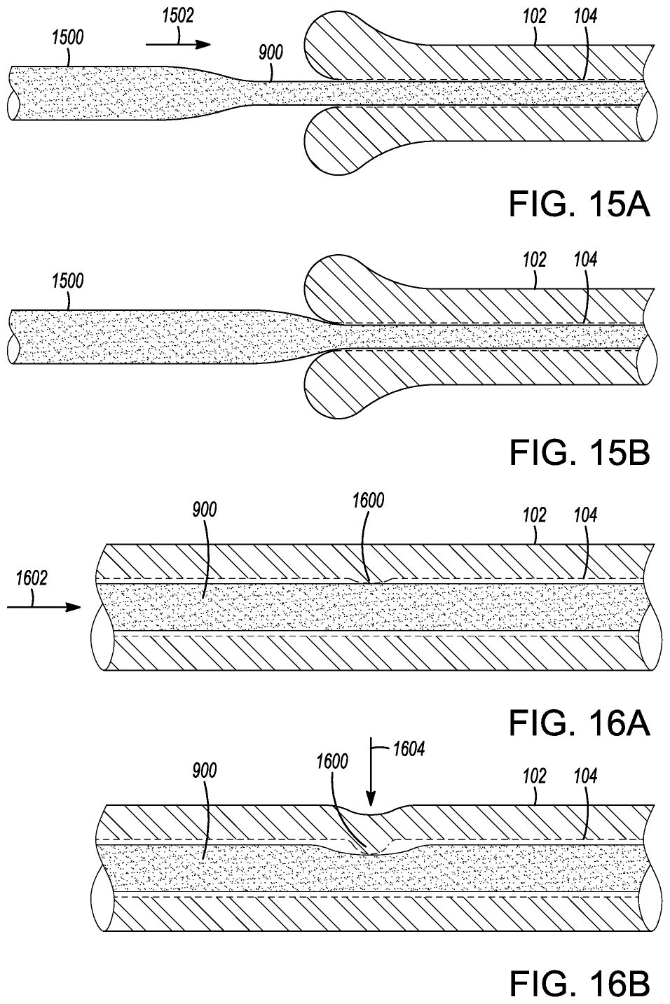

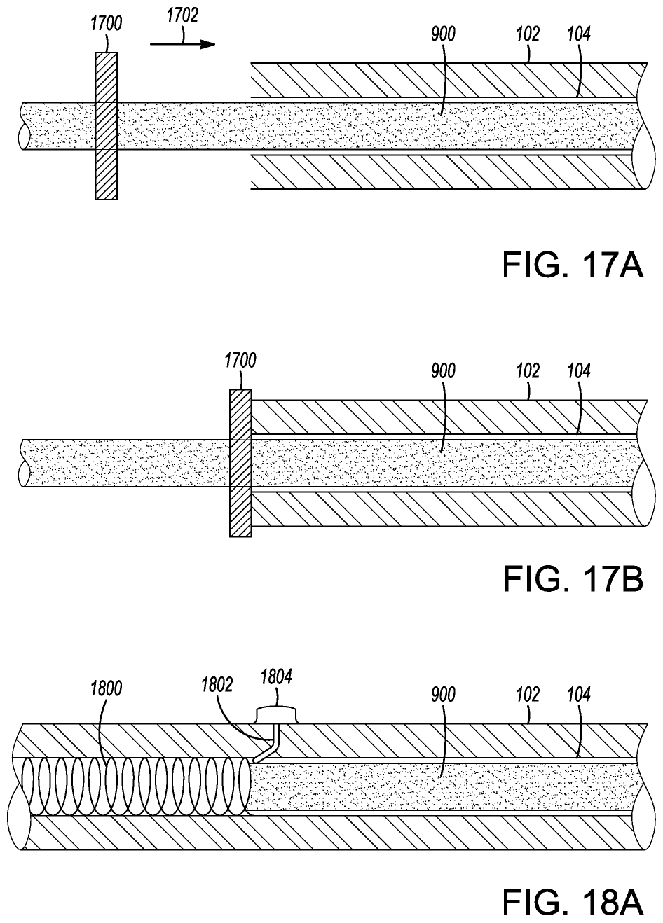

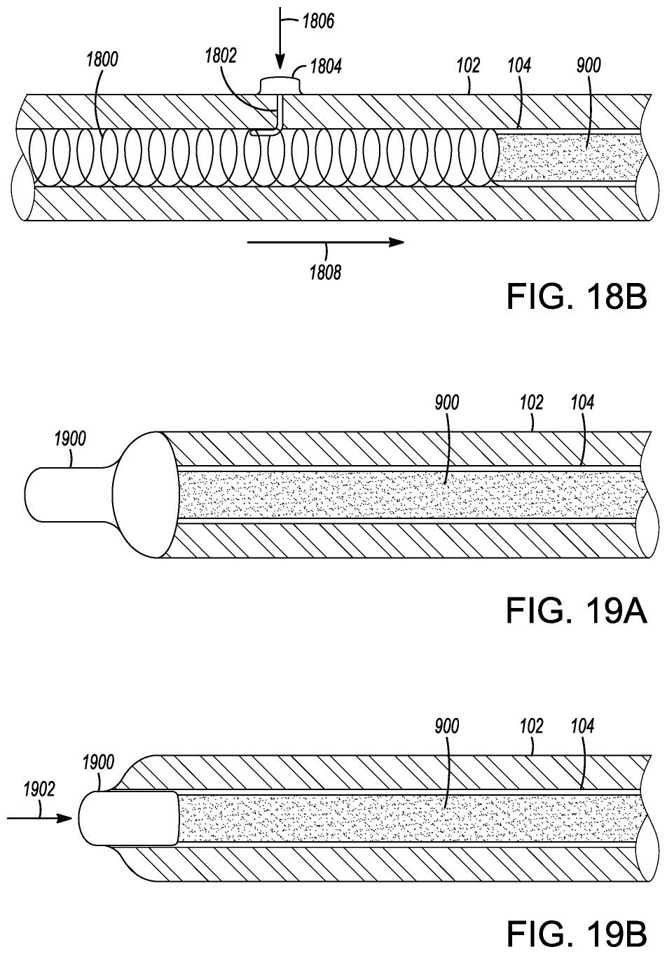

Referring to the embodiment as depicted in FIG. 3, the elongated support assembly 102 is positionable, at least in part, in a sliding relationship with the elongated flexible medical assembly 900. A sliding relationship may permit selective relative sliding between the elongated support assembly 102 and the elongated flexible medical assembly 900. A sliding relationship may permit (or include) stoppage of relative sliding between the elongated support assembly 102 and the elongated flexible medical assembly 900. Stoppage may be achieved by the embodiments depicted in FIG. 13, FIG. 14, FIG. 15, FIG. 16, FIG. 17, FIG. 18, or FIG. 19. The elongated support assembly 102 is configured to support (increase, at least in part, the stiffness of), at least in part, the elongated flexible medical assembly 900 (and of the elongated ancillary medical assembly 800) that is after the elongated support assembly 102 is positioned, at least in part, in a sliding relationship with the elongated flexible medical assembly 900. The elongated support assembly 102 is, at least in part, selectively maneuverable via the elongated ancillary medical assembly 800 toward a distal portion 802 of the elongated ancillary medical assembly 800. The elongated flexible medical assembly 900 may include a puncture device, a radiofrequency puncture device configured to puncture the interatrial septum, and any equivalent thereof. The elongated ancillary medical assembly 800 may include a medical accessory device, such as a sheath, a dilator, etc., and any equivalent thereof.

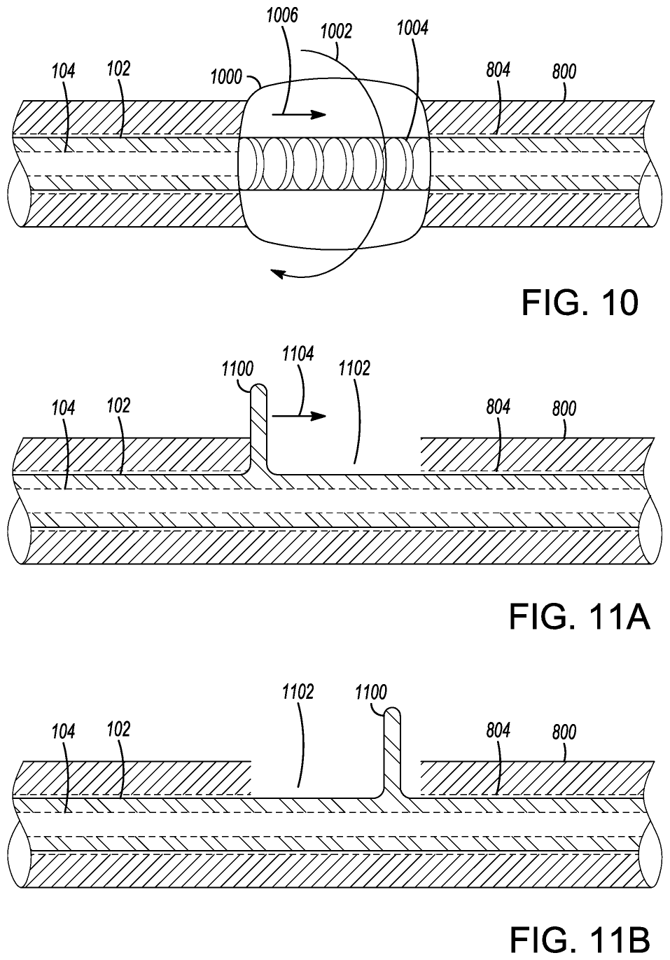

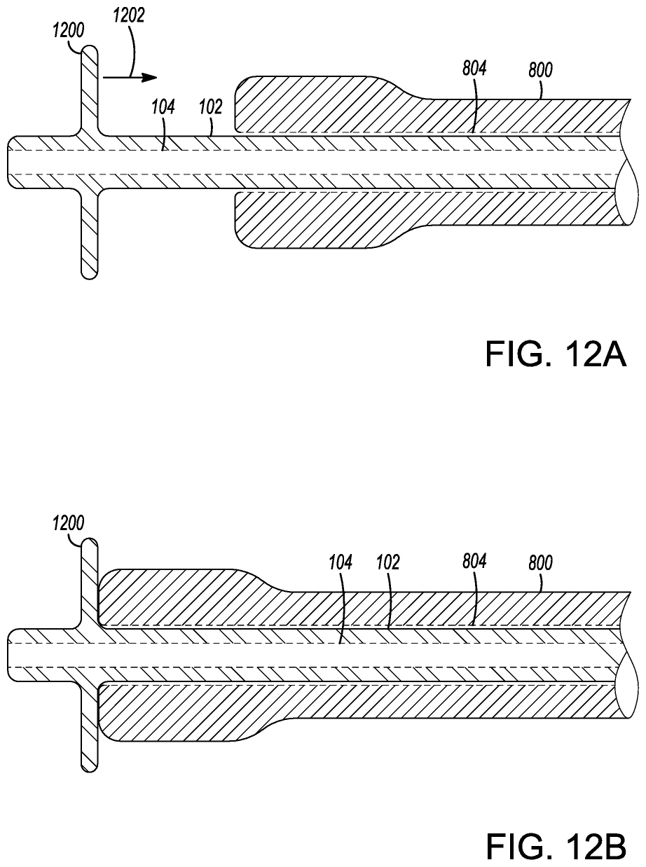

Referring to the embodiment as depicted in FIG. 3, there is provided a method of using the elongated flexible medical assembly 900, the elongated ancillary medical assembly 800 and the elongated support assembly 102. The method includes positioning, at least in part, the elongated support assembly 102 in a sliding relationship with the elongated flexible medical assembly 900. The method also includes supporting, at least in part, the elongated flexible medical assembly 900 via the elongated support assembly 102 positioned, at least in part, in a sliding relationship with the elongated flexible medical assembly 900. The method also includes selectively maneuvering, at least in part, the elongated support assembly 102 via the elongated ancillary medical assembly 800 toward a distal portion 802 of the elongated ancillary medical assembly 800. The elongated support assembly may be held in place by the embodiments depicted in FIG. 10, FIG. 11, or FIG. 12.

Referring to the embodiment as depicted in FIG. 3, the elongated support assembly 102 is configured (preferably) to increase, at least in part, stiffness of the elongated flexible medical assembly 900 and elongated ancillary medical assembly 800.

Referring to the embodiment as depicted in FIG. 3, the elongated support assembly 102 defines (preferably) a support lumen 104 extending therealong. The support lumen 104 is configured to receive the elongated flexible medical assembly 900.

Referring to the embodiments as depicted in FIG. 3 and FIG. 4, the elongated support assembly 102 is (preferably), at least in part, selectively maneuverable along with (in unison with, in a cooperative relationship with) the elongated flexible medical assembly 900 that is supported by the elongated support assembly 102 via the elongated ancillary medical assembly 800 toward the distal portion 802.

Referring to the embodiments as depicted in FIG. 3 and FIG. 4, the elongated flexible medical assembly 900 and the elongated support assembly 102 are (preferably) extendable, in unison, outwardly away from, at least in part, the distal portion 802.

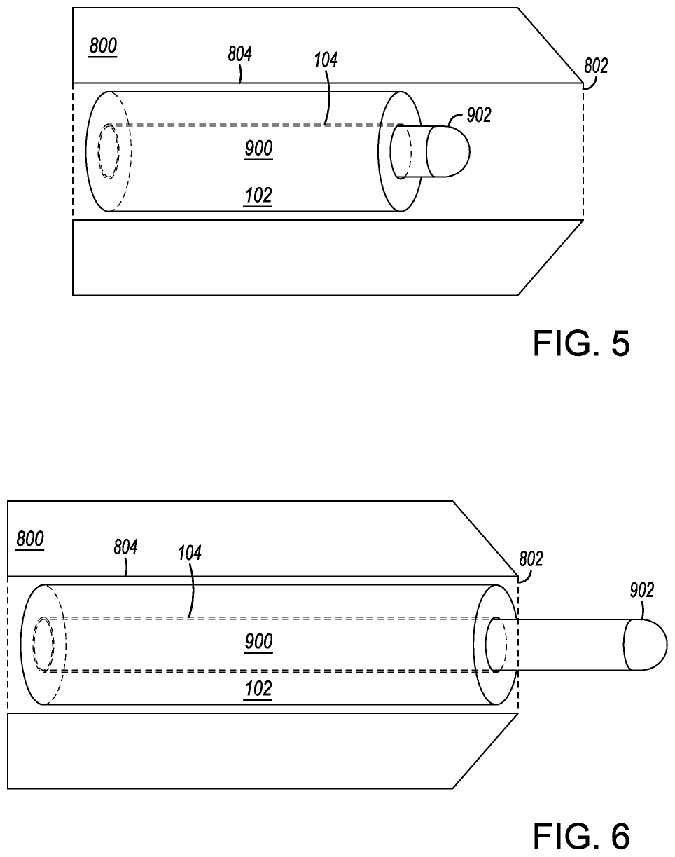

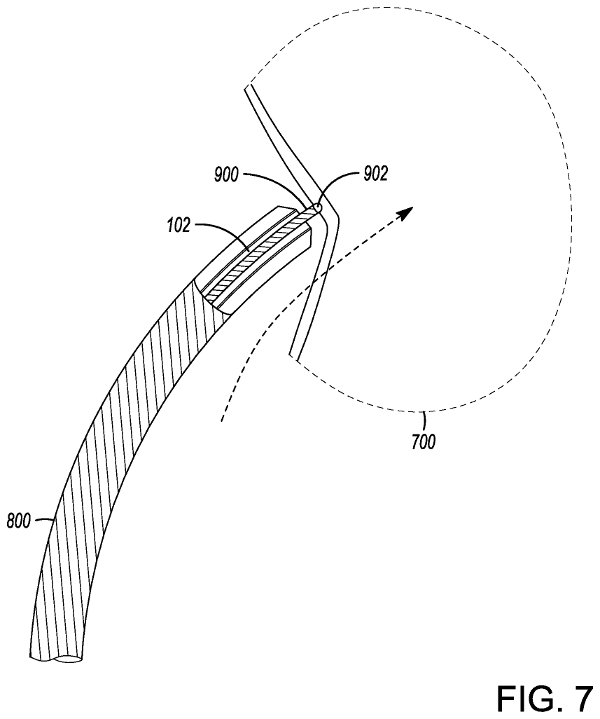

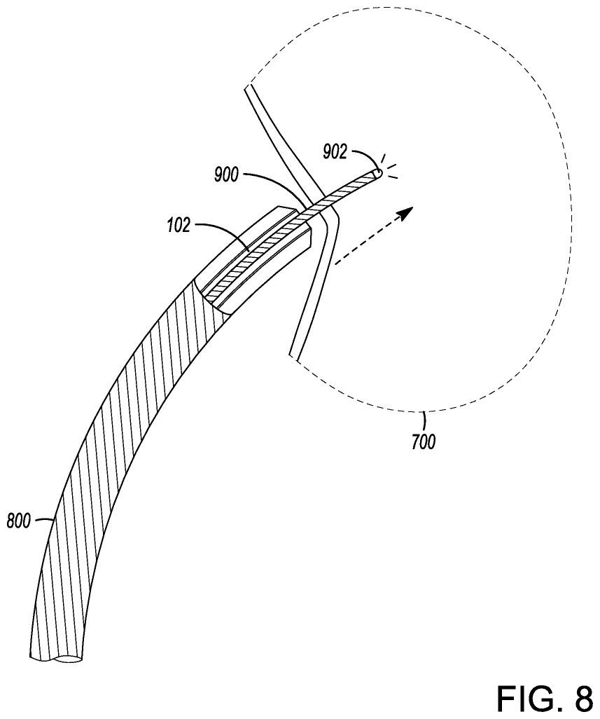

Referring to the embodiments as depicted in FIG. 5 and FIG. 6, the elongated support assembly 102 is (preferably), at least in part, selectively maneuverable along, at least in part, toward the distal portion 802, while the elongated flexible medical assembly 900 remains stationary relative to the elongated support assembly 102, and while the elongated support assembly 102, in use, continues to support, at least in part, the elongated flexible medical assembly 900.

Referring to the embodiments as depicted in FIG. 5 and FIG. 6, the elongated support assembly 102 is configured (preferably) to remain stationary relative to the elongated flexible medical assembly 900 while the elongated flexible medical assembly 900, in use, is selectively maneuverable toward the distal portion 802, and while the elongated support assembly 102, in use, continues to support, at least in part, the elongated flexible medical assembly 900.

Referring to the embodiment as depicted in FIG. 6, the elongated support assembly 102 is (preferably), at least in part, configured to remain within the elongated ancillary medical assembly 800 while the elongated flexible medical assembly 900, in use, is selectively extended outwardly away from the distal portion 802. The elongated support assembly 102 may be held within the elongated ancillary medical assembly 800 by the embodiments shown in FIG. 10, FIG. 11, or FIG. 12.

Referring to the embodiment as depicted in FIG. 7, the flexible medical assembly 900 (or the distal puncture device 902) is used to probe and/or identify a desired biological location on the biological feature 700 (such as the interatrial septum) to puncture (to form a puncture) therethrough. The flexible medical assembly 900 and the elongated support assembly 102 are used in conjunction with the ancillary medical assembly 800 (such as a sheath and/or a dilator, etc.). The flexible medical assembly 900 (or the distal puncture device 902) is positioned inside of the elongated support assembly 102.

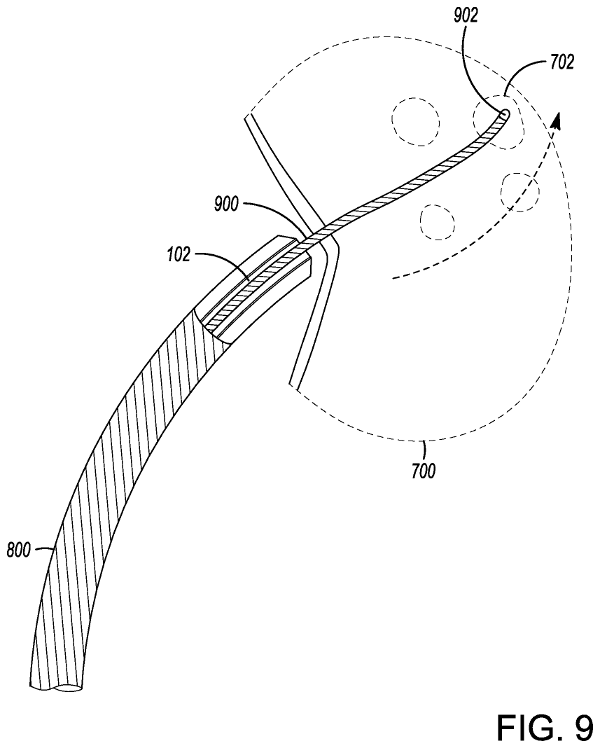

Referring to the embodiment as depicted in FIG. 8, the flexible medical assembly 900, in use, punctures through the biological feature 700 (the interatrial septum) when the distal puncture device 902 (such as once the radiofrequency energy) is activated and when the distal puncture device 902 (distal tip electrode) is positioned accordingly. With the distal puncture device 902 of the flexible medical assembly 900 positioned in the biological feature 700 (the left atrium of the heart), the distal puncture device 902 may be further deployed to secure access to the left atrial zone, etc.

Referring to the embodiment as depicted in FIG. 9, the flexible medical assembly 900 with the distal puncture device 902 is embedded in the biological feature (such as one of the pulmonary veins 702), securing left atrial access.

The following workflow steps may be employed with the elongated support assembly 102. Referring to the embodiment of FIG. 3, a first step includes inserting the elongated support assembly 102 into the ancillary medical assembly 800. Referring to the embodiment of FIG. 3, a second step includes inserting the flexible medical assembly 900 into the elongated support assembly 102 while the elongated support assembly 102 is positioned inside the ancillary medical assembly 800. Referring to the embodiment of FIG. 7, a third step includes contacting the biological feature 700 (such as the interatrial septum) with the flexible medical assembly 900 at the desired biological location to be crossed (that is, punctured through). Referring to the embodiments of FIG. 7 and FIG. 8, a fourth step includes applying radiofrequency energy to the distal puncture device 902 of the flexible medical assembly 900. Referring to the embodiments of FIG. 8, a fifth step includes advancing the flexible medical assembly 900 (from the elongated ancillary medical assembly 800) into the biological feature 700 (such as, the left atrium and secure access therein).

Referring to the embodiment as depicted in FIG. 10 (an axial cross-sectional side view), a rotatable device 1000 is configured to control (adjust, stop, prevent) movement (a slide movement or a slide relationship) between the elongated support assembly 102 and the elongated flexible medical assembly 900. The rotatable device 1000 is located (positioned) at the proximal end of the elongated ancillary medical assembly 800. The rotatable device 1000 is configured to be rotated (along the direction indicated by arrow 1002). The rotatable device 1000 is configured to be threaded with (threadably coupled to, threadably engage with) a contact portion of the elongated support assembly 102. The rotatable device 1000 includes threads 1004 configured to threadably engage the outer surface of the elongated support assembly 102. The rotatable device 1000 is configured to urge selective motion of the elongated support assembly 102 (that is, movement relative to the elongated ancillary medical assembly 800). It is understood that selective motion may include a forward motion and/or a backward motion along the direction of arrow 1006. The rotatable device 1000 is, preferably, configured to urge a screw-driven forward and backward motion (reciprocating movement) of the elongated support assembly 102 relative to the elongated ancillary medical assembly 800. The elongated support assembly 102 may begin movement at a position located proximal to the distal portion 802, as depicted in FIG. 3 or FIG. 5. Following the rotation of the rotatable device 1000 (relative to the ancillary medical assembly 800), the elongated support assembly 102 moves forwardly until the elongated support assembly 102 emerges from the ancillary lumen 804. This is done in such a way that the elongated support assembly 102 may be positioned past the distal portion 802 (as depicted in FIG. 4), or may stop at the distal portion 802 (as depicted in FIG. 6). Since forward and backward motion (of the elongated support assembly 102) are facilitated by rotation of the rotatable device 1000, the elongated support assembly 102 does not linearly slide relative to the elongated ancillary medical assembly 800 (after the rotatable device 1000 is not made to rotate, or in the absence of rotation of the rotatable device 1000). The elongated support assembly 102 is configured to slide relative to the elongated ancillary medical assembly 800 after the rotatable device 1000 is rotated (that is, in response to rotation of the rotatable device 1000).

Referring to the embodiments as depicted in FIG. 11A and FIG. 11B, a handle 1100 is configured to control (stop, prevent) movement (a slide movement or a slide relationship) between the elongated support assembly 102 and the elongated flexible medical assembly 900. FIG. 11A and FIG. 11B depict axial cross-sectional side views. A handle 1100 is attached to the proximal end of the elongated support assembly 102. The handle 1100 extends axially from the elongated support assembly 102. The handle 1100 protrudes from a portal 1102 defined by the elongated ancillary medical assembly 800. The portal 1102 is in fluid communication with the interior of the elongated ancillary medical assembly 800. The handle 1100 is configured to be moved (pushed or pulled) to control the movement of the elongated support assembly 102 (along the direction of arrow 1104) relative to the elongated ancillary medical assembly 800. It is understood that movement or linear movement may include a forward motion and/or a backward motion, etc. Movement of the elongated support assembly 102 may begin at a position located proximal to the distal portion 802 (as depicted in FIG. 3 or FIG. 5, and FIG. 11A). Following the forward advancement (movement) of the handle 1100 (as depicted in FIG. 11B) along the direction of arrow 1104 (as depicted in FIG. 11A), the elongated support assembly 102 moves forward (preferably, until the elongated support assembly 102 emerges from an end portion of the ancillary lumen 804, etc.). This is done in such a way that the elongated support assembly 102 may be positioned past the distal portion 802 (as depicted in FIG. 4), or may stop at the distal portion 802 (as depicted in FIG. 6). There is an amount of static frictional interaction (static frictional force) between the outer surface of the elongated support assembly 102 and the elongated ancillary medical assembly 800. This is done for the case where there is no relative movement between the elongated support assembly 102 and the elongated ancillary medical assembly 800. The amount of static frictional interaction is configured to maintain the relative position between the elongated support assembly 102 and the elongated ancillary medical assembly 800 (for the case where the handle 1100 is not urged to move). The amount of static frictional interaction is configured to maintain the relative position between the elongated support assembly 102 and the elongated ancillary medical assembly 800 (in response to the handle 1100 not urging the movement of the elongated support assembly 102). In response to the handle 1100 receiving a movement force, the movement force urges the handle 1100 to overcome the amount of static frictional interaction (thereby permitting movement or relative movement of the elongated support assembly 102). The movement force (to be imparted by a user to the handle 1100) is configured to overcome the amount of static frictional interaction (static frictional force) between the elongated support assembly 102 and the elongated ancillary medical assembly 800. This is done in such a way that movement may be initiated or permitted for the elongated support assembly 102 (that is, movement relative to the elongated ancillary medical assembly 800). It will be appreciated that the sliding frictional force (between the elongated support assembly 102 and the elongated ancillary medical assembly 800) is lower than the static frictional force (between the elongated support assembly 102 and the elongated ancillary medical assembly 800). If required, an appropriate lubricant may be positioned between the elongated support assembly 102 and the elongated ancillary medical assembly 800 (to achieve the desired effect).

Referring to the embodiments as depicted in FIG. 12A and FIG. 12B, a proximal hub 1200 is configured to control (stop, prevent) movement (a slide movement or a slide relationship) between the elongated support assembly 102 and the elongated flexible medical assembly 900. FIG. 12A and FIG. 12B depict axial cross-sectional side views. The elongated support assembly 102 includes (has) the proximal hub 1200. The proximal hub 1200 extends from an outer surface of the elongated support assembly 102. The proximal hub 1200 is configured to abut (at least in part) an entrance leading into the ancillary lumen 804 of the elongated ancillary medical assembly 800 (after the elongated support assembly 102 moves toward the entrance leading into the ancillary lumen 804). For the case where the proximal hub 1200 is moved to abut or contact (at least in part) the entrance leading into the ancillary lumen 804 (as a result of the movement of the elongated support assembly 102), the elongated support assembly 102 is stopped from further movement along the ancillary lumen 804. The proximal hub 1200 is configured to be movable toward, but cannot enter into, the ancillary lumen 804. The proximal hub 1200 is movable, with the elongated support assembly 102, along the direction of arrow 1202. The proximal hub 1200 is (preferably) sized to be larger than the size of the entrance of the ancillary lumen 804 (of the elongated ancillary medical assembly 800). The elongated support assembly 102 may, for instance, begin movement at a position located proximal to the distal portion 802 (as shown in FIG. 3 or FIG. 5, and FIG. 12A).

Referring to the embodiment as depicted in FIG. 12B, following forward advancement (movement) of the proximal hub 1200, the elongated support assembly 102 is moved (forwardly by the user) until the elongated support assembly 102 emerges from the ancillary lumen 804. This is done in such a way that the elongated support assembly 102 may be positioned past the distal portion 802 (as depicted in FIG. 4) or may stop (from further movement) at a position located at the distal portion 802 (as depicted in FIG. 6). There is an amount of static frictional interaction (static frictional force) between the elongated support assembly 102 and the elongated ancillary medical assembly 800. The amount of static frictional interaction is configured to maintain the relative positions between the elongated support assembly 102 and the elongated ancillary medical assembly 800 (while movement is not imparted to the proximal hub 1200, or for the case where the proximal hub 1200 is not urged to move, etc.). A movement force, to be imparted by a user (to the proximal hub 1200), is configured to overcome the amount of static frictional interaction (static frictional force) between the elongated support assembly 102 and the elongated ancillary medical assembly 800. This is done in such a way that the elongated support assembly 102 is permitted to move (or may initiate movement relative to the elongated ancillary medical assembly 800). It will be appreciated that the sliding frictional force (between the elongated support assembly 102 and the elongated ancillary medical assembly 800) is lower than the static frictional force (between the elongated support assembly 102 and the elongated ancillary medical assembly 800).

Referring to the embodiments as depicted in FIG. 13A, FIG. 13B and FIG. 13C, a rotatable element 1300 is configured to control (stop, prevent) movement (a slide movement or a slide relationship) between the elongated support assembly 102 and the elongated flexible medical assembly 900. FIG. 13A depicts an axial cross-sectional side view. FIG. 13B and FIG. 13C depict radial cross-sectional side views taken along a cross-sectional line A-A as depicted in FIG. 13A. The rotatable element 1300 may include, for instance, a tuohy-borst adapter. The tuohy-borst adapter is known to those persons skilled in the art. The tuohy-borst adapter may include a body, a gasket, and a cap. The tuohy-borst adapter is configured to prevent the backflow of fluid. The tuohy-borst adapter is also configured to facilitate catheter introduction (the silicone valve and the cap torque around a tube or an instrument to hold the tube in place). The elongated support assembly 102 includes the rotatable element 1300. The rotatable element 1300 is positioned at the proximal end of the elongated support assembly 102. The rotatable element 1300 includes a flexible element 1302 (such as silicone, etc.) positioned proximate to (adjacent to) the support lumen 104 (of the elongated support assembly 102). The rotatable element 1300 is configured to change (reduce or increase) an amount of compression applied to the flexible element 1302. The rotatable element 1300 is configured to change an amount of compression applied from the flexible element 1302 to the support lumen 104 of the elongated support assembly 102. A change in compression (to be applied to the flexible element 1302) creates a change (increase or decrease) in the effective size (inner diameter) of the support lumen 104 (of the elongated support assembly 102). The flexible element 1302 is configured to change the effective size of the support lumen 104 of the elongated support assembly 102.

Referring to the embodiment as depicted in FIG. 13A, the support lumen 104 is open (preferably fully open) in response to a lower amount of application (or no application) of a compression force to the flexible element 1302. The rotatable element 1300 does not apply the compression force to the flexible element 1302. In response to no application of the compression force to the flexible element 1302, the elongated flexible medical assembly 900 is permitted to be (freely) advanced (at least in part) along an axial length of the support lumen 104 (extending through the elongated support assembly 102). The direction (indicated by the direction of arrow 1301) of rotation of the rotatable element 1300 is the direction for the application of a compression force to the flexible element 1302. After the compression force is applied to the flexible element 1302, the diameter (inner diameter) of the support lumen 104 is reduced (at least in part).

Referring to the embodiment as depicted in FIG. 13B, the flexible element 1302 is not compressed (is placed in the uncompressed state). For the case where the rotatable element 1300 does not compress the flexible medical assembly 900, the inner diameter of the support lumen 104 becomes larger than the diameter of the elongated flexible medical assembly 900. This is done in such a way that the elongated flexible medical assembly 900 may freely advance within (along) the elongated support assembly 102.

Referring to the embodiment as depicted in FIG. 13C, the rotatable element 1300 was actuated to compress the flexible element 1302. The rotatable element 1300 is configured to apply the compression force to the flexible element 1302. As a result (of the application of the compression force from the rotatable element 1300), the inner diameter of the support lumen 104 becomes relatively smaller (at the section or portion located adjacent to the flexible element 1302). This is done in such a way that the support lumen 104, in use, restricts movement (preferably, achieves stoppage of any movement) of the elongated flexible medical assembly 900. This is done in such a way that the flexible medical assembly 900 no longer slides (is stopped from sliding) relative to the elongated support assembly 102.

Referring to the embodiments as depicted in FIG. 14A, FIG. 14B and FIG. 14C, a slidable element 1400 is configured to control (stop, prevent) movement (a slide movement or a slide relationship) between the elongated support assembly 102 and the elongated flexible medical assembly 900. FIG. 14A and FIG. 14B depict axial cross-sectional side views. FIG. 14C depicts an overhead view or a top view. The elongated support assembly 102 includes the slidable element 1400. The slidable element 1400 is, preferably, integrated into the proximal end of the elongated support assembly 102. The slidable element 1400 is configured to selectively contact (frictionally contact) the elongated flexible medical assembly 900 (after the elongated flexible medical assembly 900 is received in the elongated support assembly 102). This is done in such a way that the slidable element 1400 (in use) contacts (abuts) the outer surface of the elongated flexible medical assembly 900. The slidable element 1400 is also configured to selectively move the elongated flexible medical assembly 900 (this is done after the slidable element 1400, in use, selectively contacts or frictionally contacts the elongated flexible medical assembly 900, as indicated along the direction of arrow 1402, as depicted in FIG. 14A). The slidable element 1400 is configured to be moved (by the user) along an axial length of the elongated support assembly 102 (while the user maintains contact with the slidable element 1400). The elongated flexible medical assembly 900 moves in response to the slidable element 1400 moving (advancing, retracting, etc.) along, or on, the elongated support assembly 102 (while the elongated flexible medical assembly 900 and the slidable element 1400 remain in contact with each other).

Referring to the embodiment as depicted in FIG. 14A, the elongated flexible medical assembly 900 is (fully) retracted (in response to the movement of the slidable element 1400). This condition may correspond to the distal configuration (as depicted in FIG. 5). Movement of the slidable element 1400 (along the direction of arrow 1402) is done in such a way that the elongated flexible medical assembly 900 is (fully) advanced or moved (as depicted in FIG. 14B).

Referring to the embodiment as depicted in FIG. 14B, there is depicted the full advancement of the elongated flexible medical assembly 900 via the sliding element. This condition may correspond to the distal configuration (as depicted in FIG. 6).

Referring to the embodiment as depicted in FIG. 14C, there is depicted an overhead view of the slidable element 1400. The slidable element 1400 may be forwardly to thereby advance (move) the elongated flexible medical assembly 900. Since the elongated flexible medical assembly 900 cannot move without the movement of the slidable element 1400, stoppage of the relative sliding between the elongated support assembly 102 and the elongated flexible medical assembly 900 is achieved when the slidable element 1400 is not manipulated.

Referring to the embodiments as depicted in FIG. 15A and FIG. 15B, a proximal tapered section 1500 is configured to control (stop, prevent) movement (a slide movement or a slide relationship) between the elongated support assembly 102 and the elongated flexible medical assembly 900. FIG. 15A and FIG. 15B depict axial cross-sectional side views. The elongated flexible medical assembly 900 includes (possesses) the proximal tapered section 1500. The proximal tapered section 1500 is positioned on, and extends from, an outer surface (outer diameter) of the elongated flexible medical assembly 900. The outer diameter of the proximal tapered section 1500 is larger than (exceeds) the inner diameter of (the support lumen 104) of the elongated support assembly 102.

Referring to the embodiment as depicted in FIG. 15A, the elongated flexible medical assembly 900 is advanced (moved along the direction of arrow 1502) through the elongated support assembly 102 along arrow 1502.

Referring to the embodiment as depicted in FIG. 15B, the proximal tapered section 1500 (of the elongated flexible medical assembly 900) is moved to (eventually) abut (interact or contact) the proximal end of the elongated support assembly 102. For the case where the proximal tapered section 1500 (in use) contacts (is moved to contact), or abuts, the end portion of the elongated support assembly 102, further advancement of the elongated flexible medical assembly 900 is stopped. This is done in such a way that the elongated flexible medical assembly 900 may not (cannot) proceed further along the direction of arrow 1502, as depicted in FIG. 15A. For the case where the proximal tapered section 1500 is moved to abut or contact the elongated support assembly 102, further advancement (of the elongated flexible medical assembly 900) cannot occur (into the interior of the elongated support assembly 102). This is done in such a way that there is a stoppage of the relative sliding (movement) between the elongated support assembly 102 and the elongated flexible medical assembly 900.

Referring to the embodiments as depicted in FIG. 16A and FIG. 16B, a flexible region 1600 is configured to control (stop, prevent) movement (a slide movement or a slide relationship) between the elongated support assembly 102 and the elongated flexible medical assembly 900. FIG. 16A and FIG. 16B depict axial cross-sectional side views. The flexible region 1600 is positioned on (in) a proximal section of the elongated support assembly 102. The elongated support assembly 102 is configured to support the flexible region 1600. The flexible region 1600 is configured to be depressed (or compressed by the user, etc.).

Referring to the embodiment as depicted in FIG. 16A, the elongated flexible medical assembly 900 is movable (freely movable, along the direction of arrow 1602) within the elongated support assembly 102. The flexible region 1600 is configured to remain undepressed for the case where the user has not applied a depression force to the flexible region 1600 (as depicted in FIG. 16A). The elongated flexible medical assembly 900 is movable (along the direction indicated by arrow 1602) within or along the elongated support assembly 102 while the flexible region 1600 remains undepressed (uncompressed), as depicted in FIG. 16A. The elongated flexible medical assembly 900 is not movable within or along the elongated support assembly 102 while the flexible region 1600 remains depressed (compressed), as depicted in FIG. 16B (since the user is applying the depression force to the flexible region 1600).

Referring to the embodiment as depicted in FIG. 16B, the flexible region 1600 has been pushed or moved (along the direction of arrow 1604), and the flexible region 1600 is placed in the depressed state. In the depressed state, the flexible region 1600 interacts with (selectively contacts) the elongated flexible medical assembly 900 that is positioned inside the lumen (the support lumen 104) of the elongated support assembly 102. This is done in such a way that the elongated flexible medical assembly 900 is stopped from further movement along the support lumen 104. Static friction (contact friction) is created between the elongated flexible medical assembly 900 and the elongated support assembly 102 (when there is no relative movement therebetween). The static friction is configured to prevent further movement of the elongated flexible medical assembly 900 (such as along the direction of arrow 1602, as depicted in FIG. 16A). Static friction between the elongated flexible medical assembly 900 and the elongated support assembly 102 is configured to prevent further movement of the elongated flexible medical assembly 900 after the flexible region 1600 is not depressed. The static friction provided by activation (depression) of the flexible region 1600 achieves stoppage of the relative sliding between the elongated support assembly 102 and the elongated flexible medical assembly 900.

Referring to the embodiments as depicted in FIG. 17A and FIG. 17B, a block device 1700 is configured to control (stop, prevent) movement (a slide movement or a slide relationship) between the elongated support assembly 102 and the elongated flexible medical assembly 900. FIG. 17A and FIG. 17B depict axial cross-sectional side views. The block device 1700 is fixed (affixed) to a portion of the elongated flexible medical assembly 900. The outer diameter of the block device 1700 is greater than (exceeds) the inner diameter (the support lumen 104) of the elongated support assembly 102. The block device 1700 is configured to be not insertable into the support lumen 104.

Referring to the embodiment as depicted in FIG. 17A, the elongated flexible medical assembly 900 is advanced into the support lumen 104 of the elongated support assembly 102, and the elongated flexible medical assembly 900 is movable (along the direction of arrow 1702).

Referring to the embodiment as depicted in FIG. 17B, the block device 1700 is moved (along the direction of arrow 1702, as depicted in FIG. 17A). This is done in such a way that the block device 1700 reaches (contacts, abuts) the proximal end of the elongated support assembly 102. The elongated flexible medical assembly 900 is prevented from further advancement into the elongated support assembly 102 after the block device 1700 is moved to contact (abut) the elongated support assembly 102 (since the block device 1700 cannot move into the interior of the elongated support assembly 102). The block device 1700 is configured to stop relative sliding movement between the elongated support assembly 102 and the elongated flexible medical assembly 900.

Referring to the embodiments as depicted in FIG. 18A and FIG. 18B, a biasing device 1800 (such as a spring device, etc.) is configured to control (stop, prevent) movement (a slide movement or a slide relationship) between the elongated support assembly 102 and the elongated flexible medical assembly 900. FIG. 18A and FIG. 18B depict axial cross-sectional side views. The biasing device 1800 is positioned (located) proximal to the elongated flexible medical assembly 900. The biasing device 1800 is configured to abut the end portion of the elongated flexible medical assembly 900. The elongated support assembly 102 includes a stopper 1802 positioned in the support lumen 104 (of the elongated support assembly 102). The elongated support assembly 102 also includes a depression device 1804 positioned on the outer surface of the elongated support assembly 102. The stopper 1802 is coupled to the depression device 1804. The biasing device 1800 is configured to contact the stopper 1802 and be compressed by the stopper 1802 (in response to the application of a compression force to the biasing device 1800). The stopper 1802 is configured to selectively move away from the biasing device 1800 (in response to user activation of the depression device 1804).

Referring to the embodiment as depicted in FIG. 18A, the biasing device 1800 is in a compressed state, and the elongated flexible medical assembly 900 is fully retracted. This case may correspond to the distal configuration (as depicted in FIG. 5).

Referring to the embodiment as depicted in FIG. 18B, the user applies an activation force to the depression device 1804 (along the direction of arrow 1806). This is done in such a way that the stopper 1802 is moved so that the biasing device 1800 may be released (to decompress the biasing device 1800) after the stopper 1802 has been removed (or moved aside). Release of the biasing device 1800 (from the compressed state) thereby urges the elongated flexible medical assembly 900 to move forwardly (preferably, until the biasing device 1800 reaches an equilibrium length, as depicted in FIG. 18B). User activation of the depression device 1804 (for actuation of the stopper 1802) causes the release of the biasing device 1800. After the stopper 1802 is released from the biasing device 1800, the biasing device 1800 imparts forward advancement to the elongated flexible medical assembly 900 until the biasing device 1800 reaches an equilibrium length (and preferably no further advancement occurs). This case may correspond to the distal configuration (as depicted in FIG. 6). The biasing device 1800 travels forward until the biasing device 1800 has reached its equilibrium length (since the elongated flexible medical assembly 900 cannot move without the release of the biasing device 1800). The biasing device 1800 extends along the direction of arrow 1808 when the biasing device 1800 is released. Stoppage of the relative sliding between the elongated support assembly 102 and the elongated flexible medical assembly 900 occurs after (preferably) the biasing device 1800 reaches the equilibrium length.

Referring to the embodiments as depicted in FIG. 19A and FIG. 19B, an actuatable plunger 1900 is configured to control (stop, prevent) movement (a slide movement or a slide relationship) between the elongated support assembly 102 and the elongated flexible medical assembly 900. FIG. 19A and FIG. 19B depict axial cross-sectional side views. The actuatable plunger 1900 is located at the proximal end of the elongated support assembly 102. The actuatable plunger 1900 is located proximal to the elongated flexible medical assembly 900. The actuatable plunger 1900 is configured (preferably) to function the same way as a known click pen (writing instrument).

Referring to the embodiment as depicted in FIG. 19A, the actuatable plunger 1900 is placed in a fully retracted state. The elongated flexible medical assembly 900 is fully retracted in the elongated support assembly 102 in this configuration. This may correspond to the distal configuration shown in FIG. 5.

Referring to the embodiment as depicted in FIG. 19B, activation of the actuatable plunger 1900 is possible by moving the actuatable plunger 1900 along the direction of arrow 1902. Depression of the actuatable plunger 1900 pushes the elongated flexible medical assembly 900 proximally This is done in such a way that the elongated flexible medical assembly 900 may move (forwardly relative to the elongated support assembly 102). This may correspond to the distal configuration shown in FIG. 6. Preferably, the elongated flexible medical assembly 900 does not move without the depression of the actuatable plunger 1900. Preferably, the elongated flexible medical assembly 900 is configured to move in response to the depression of the actuatable plunger 1900. The actuatable plunger 1900 may move a prescribed distance. Stoppage of the relative sliding between the elongated support assembly 102 and the elongated flexible medical assembly 900 occurs when the actuatable plunger 1900 is fully depressed.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus for use with an elongated flexible medical assembly and an elongated ancillary medical assembly, the apparatus comprising:
an elongated support assembly being positionable, at least in part, in sliding relationship with the elongated flexible medical assembly;
the elongated support assembly configured to support, at least in part, the elongated flexible medical assembly after the elongated support assembly is positioned, at least in part, in sliding relationship with the elongated flexible medical assembly; and
the elongated support assembly being, at least in part, selectively maneuverable via the elongated ancillary medical assembly toward a distal portion of the elongated ancillary medical assembly.

2. (canceled)

3. The apparatus of claim 1, wherein:
the elongated support assembly is, at least in part, selectively maneuverable along, at least in part, toward the distal portion, while the elongated flexible medical assembly remains stationary relative to the elongated support assembly, and while the elongated support assembly, in use, continues to support, at least in part, the elongated flexible medical assembly.

4. The apparatus of claim 1, wherein:
the elongated support assembly is configured to remain stationary relative to the elongated flexible medical assembly while the elongated flexible medical assembly, in use, is selectively maneuverable toward the distal portion, and while the elongated support assembly, in use, continues to support, at least in part, the elongated flexible medical assembly.

5. The apparatus of claim 1, wherein:
the elongated support assembly is, at least in part, configured to remain within the elongated ancillary medical assembly while the elongated flexible medical assembly, in use, is selectively extended outwardly away from the distal portion.

6. The apparatus of claim 1, wherein:
the elongated support assembly is configured to increase, at least in part, stiffness of the elongated flexible medical assembly and of the elongated ancillary medical assembly.

7. The apparatus of claim 1, wherein:
the elongated support assembly is configured to increase, at least in part, stiffness of the elongated flexible medical assembly and of the elongated ancillary medical assembly.

8.-11. (canceled)

12. The apparatus of claim 1, wherein:
the elongated ancillary medical assembly includes a dilator assembly.

13.-14. (canceled)

15. The apparatus of claim 1, wherein:
the elongated flexible medical assembly includes:
a distal puncture device configured to puncture a biological feature.

16.-19. (canceled)

20. The apparatus of claim 1, wherein:
a rotatable device is configured to control movement between the elongated support assembly and the elongated flexible medical assembly; the rotatable device is located at a proximal end of the elongated ancillary medical assembly;
the rotatable device is configured to be rotated;
the rotatable device is configured to threadably engage an outer surface of the elongated support assembly; and
the rotatable device is configured to urge selective motion of the elongated support assembly.

21. The apparatus of claim 1, wherein:
a handle is configured to control movement between the elongated support assembly and the elongated flexible medical assembly;
the handle is attached to a proximal end of the elongated support assembly;
the handle extends axially from the elongated support assembly 102;
the handle protrudes from a portal of the elongated ancillary medical assembly;
the portal is in fluid communication with an interior of the elongated ancillary medical assembly;
the handle is configured to be moved to control linear movement of the elongated support assembly;
there is an amount of static frictional interaction between an outer surface of the elongated support assembly and an inner surface of the elongated ancillary medical assembly in such a way that there is a lack of relative movement between the elongated support assembly and the elongated ancillary medical assembly; and
the amount of static frictional interaction is configured to maintain relative position between the elongated support assembly and the elongated ancillary medical assembly in response to the handle not urging the movement of the elongated support assembly, and in response to the handle receiving a movement force, the movement force overcomes the amount of static frictional interaction, to permit movement of the elongated support assembly.

22. The apparatus of claim 1, wherein:
a proximal hub is configured to control movement between the elongated support assembly and the elongated flexible medical assembly;
the elongated support assembly includes the proximal hub;
the proximal hub extends from an outer surface of the elongated support assembly;
the proximal hub is configured to abut, at least in part, an entrance leading into an ancillary lumen of the elongated ancillary medical assembly in response to the elongated support assembly moving toward the entrance leading into the ancillary lumen in such a way that after the proximal hub becomes abutted, at least in part, to the entrance leading into the ancillary lumen as a result of movement of the elongated support assembly, the elongated support assembly is stopped from further movement along the ancillary lumen;
the proximal hub is configured to be movable toward, but cannot enter into, the ancillary lumen; and
the proximal hub is sized to be larger than the entrance of the ancillary lumen of the elongated ancillary medical assembly.

23. The apparatus of claim 1, wherein:
a rotatable element is configured to control movement between the elongated support assembly and the elongated flexible medical assembly;
the elongated support assembly includes the rotatable element;
the rotatable element is positioned at a proximal end of the elongated support assembly;
the rotatable element includes a flexible element that is positioned proximate to a support lumen of the elongated support assembly;
the rotatable element is configured to change an amount of compression applied form the flexible element to the support lumen of the elongated support assembly;
the flexible element is configured to change an effective size of the support lumen of the elongated support assembly.

24. The apparatus of claim 1, wherein:
a slidable element is configured to control movement between the elongated support assembly and the elongated flexible medical assembly;
the elongated support assembly includes the slidable element;
the slidable element is configured to selectively frictionally contact the elongated flexible medical assembly after the elongated flexible medical assembly is received in the elongated support assembly, and the slidable element, in use, contacts an outer surface of the elongated flexible medical assembly;
the slidable element is also configured to selectively move the elongated flexible medical assembly after the slidable element, in use, selectively frictionally contacts the elongated flexible medical assembly;
the slidable element is configured to be movable along an axial length of the elongated support assembly; and
the elongated flexible medical assembly moves in response to the slidable element moving along the elongated support assembly, while the elongated flexible medical assembly and the slidable element remain in contact with each other.

25. The apparatus of claim 1, wherein:
a proximal tapered section is configured to control movement between the elongated support assembly and the elongated flexible medical assembly;
the elongated flexible medical assembly includes the proximal tapered section; the proximal tapered section is positioned on, and extends from, an outer surface of the elongated flexible medical assembly;
an outer diameter of the proximal tapered section is larger than an inner diameter of a support lumen of the elongated support assembly; and
the proximal tapered section is movable to abut a proximal end of the elongated support assembly in such a way that the proximal tapered section, in use, contacts an end portion of the elongated support assembly, and further advancement of the elongated flexible medical assembly is stopped so that the elongated flexible medical assembly does not proceed further.

26. The apparatus of claim 1, wherein:
a flexible region is configured to control movement between the elongated support assembly and the elongated flexible medical assembly;
the flexible region is positioned in a proximal section of the elongated support assembly;
the elongated support assembly is configured to support the flexible region; and
the flexible region is configured to be depressed;
the elongated flexible medical assembly is movable within the elongated support assembly after the flexible region has not been depressed;
in a depressed state of the flexible region, the flexible region interacts with the elongated flexible medical assembly that is positioned inside a support lumen of the elongated support assembly in such a way that the elongated flexible medical assembly is stopped from further movement along the support lumen; and
static friction is created during interaction between the elongated flexible medical assembly and the elongated support assembly to prevent further movement of the elongated flexible medical assembly while the flexible region remains undepressed.

27. The apparatus of claim 1, wherein:
a block device is configured to control movement between the elongated support assembly and the elongated flexible medical assembly;
the block device is fixed to a portion of the elongated flexible medical assembly;
an outer diameter of the block device is greater than an inner diameter of a support lumen of the elongated support assembly;
the block device is configured to be not insertable into the support lumen; and
the block device is movable in such a way that the block device contacts a proximal end of the elongated support assembly, and the elongated flexible medical assembly is prevented from further advancement into the elongated support assembly after the block device is moved to contact the elongated support assembly.

28. The apparatus of claim 1, wherein:
a biasing device is configured to control movement between the elongated support assembly and the elongated flexible medical assembly;
the biasing device is positioned proximal to the elongated flexible medical assembly; and
the biasing device is configured to abut an end portion of the elongated flexible medical assembly;
the elongated support assembly includes a stopper positioned in a support lumen of the elongated support assembly;
the elongated support assembly also includes a depression device positioned on an outer surface of the elongated support assembly;
the stopper is coupled to the depression device;
the biasing device is configured to contact, and become compressed by, the stopper in response to an application of a compression force to the biasing device;
the stopper is configured to selectively move away from the biasing device by user activation of the depression device;
user activation of the depression device is done in such a way that the stopper is moved so that the biasing device is released; and
release of the biasing device, from a compressed state, urges the elongated flexible medical assembly to move forwardly, and after the stopper is released from the biasing device, the biasing device imparts forward advancement to the elongated flexible medical assembly.

29. The apparatus of claim 1, wherein:
an actuatable plunger is configured to control movement between the elongated support assembly and the elongated flexible medical assembly;
the actuatable plunger is located at a proximal end of the elongated support assembly;
the actuatable plunger is located proximal to the elongated flexible medical assembly; and
depression of the actuatable plunger pushes the elongated flexible medical assembly proximally, causing forward advancement of the elongated flexible medical assembly.

30. The apparatus of claim 1, wherein:
the elongated support assembly includes a first region having a first stiffness;
the first region of the elongated support assembly is configured to be positioned within the ancillary lumen located proximate to a curved section at a distal end of the ancillary medical assembly;
the elongated support assembly includes a second region having a second stiffness; the second region of the elongated support assembly is configured to be positioned at a curved region of the ancillary medical assembly and beyond; and
the first region of the elongated support assembly is relatively stiffer than the second region of the elongated support assembly in such a way that distortion of any curvature at the distal end of the ancillary medical assembly is prevented after (A) the first region of the elongated support assembly is positioned within the ancillary lumen located proximate to a curved section at a distal end of the ancillary medical assembly, and (B) the second region of the elongated support assembly is positioned at a curved region of the ancillary medical assembly and beyond.
